(19) United States
(12) Patent Application Publication
(10) Pub. No.: US 2017/0360826 A1
(43) Pub. Date: Dec. 21, 2017

(54) METHODS FOR TREATING ALCOHOL WITHDRAWAL

(71) Applicants: Antti O. Patiala, San Antonio, TX (US); Reijo Ojutkangas, ESPOO (FI)

(72) Inventors: Antti O. Patiala, San Antonio, TX (US); Reijo Ojutkangas, ESPOO (FI)

(21) Appl. No.: 15/183,366

(22) Filed: Jun. 15, 2016

(51) Int. Cl. A61K 33/00; A61M 16/12; A61M 16/06; A61M 16/10

(52) U.S. Cl. CPC A61K 33/00; A61M 16/104; A61M 16/122; A61M 16/0672; A61M 2202/0208; A61M 2202/0283

(57) ABSTRACT

This disclosure includes a method for treating a patient suffering from alcohol withdrawal. The method comprises administering an effective amount of oxygen to the patient and administering an effective amount of nitrous oxide to the patient. In some embodiments, the effective amount of oxygen and the effective amount of nitrous oxide are administered to the patient via a nitrous oxide sedation system.

### BACKGROUND

### Field

The present invention relates generally to treatments, and more particularly to methods for treating alcohol withdrawal.

### Description of Related Art

The brain is the body's control center and manages every move the body makes. When a person consumes alcohol it effects brain functionality. The effects can result in long-term changes, which can occur when a person becomes addicted to alcohol. In some instances, a person becomes addicted to alcohol when his or her brain adjusts to the way alcohol alters the brain so that drinking becomes necessary in order to function. In this regard, when the brain is withdrawn from the effects of alcohol the brain becomes too excited and the brain needs the slowing effects of alcohol in order to function properly. Thus, there is a need for a method to effectively treat the effects from alcohol withdrawal in a patient.

### SUMMARY

The present disclosure includes a method for treating a patient suffering from alcohol withdrawal. The method can include administering an effective amount of oxygen to the patient and administering an effective amount of nitrous oxide to the patient. In some embodiments, administering the effective amount of oxygen to the patient comprises administering a first effective amount of oxygen to the patient for a first predetermined amount of time, and administering the effective amount of nitrous oxide to the patient comprises administering a first effective amount of nitrous oxide to the patient for the first predetermined amount of time.

After administering the first effective amount of oxygen and the first effective amount of nitrous oxide to the patient for the first predetermined amount of time, some methods include administering a second effective amount of oxygen and a second effective amount of nitrous oxide to the patient for a second predetermined amount of time. Even still, after administering the second effective amount of oxygen and the second effective amount of nitrous oxide to the patient for the second predetermined amount of time, some methods include administering a third effective amount of oxygen and a third effective amount of nitrous oxide to the patient for a third predetermined amount of time.

Even still, after administering the third effective amount of oxygen and the third effective amount of nitrous oxide to the patient for the third predetermined amount of time, methods can include administering a fourth effective amount of oxygen and a fourth effective amount of nitrous oxide to the patient for a fourth predetermined amount of time. After administering the fourth effective amount of oxygen and the fourth effective amount of nitrous oxide to the patient for the fourth predetermined amount of time, some methods include administering a fifth effective amount of oxygen and a fifth effective amount of nitrous oxide to the patient for a fifth predetermined amount of time.

After administering the fifth effective amount of oxygen and the fifth effective amount of nitrous oxide to the patient for the fifth predetermined amount of time, some methods include administering a sixth effective amount of oxygen and a sixth effective amount of nitrous oxide to the patient for a sixth predetermined amount of time. After administering the sixth effective amount of oxygen and the sixth effective amount of nitrous oxide to the patient for the sixth predetermined amount of time, some methods include administering a seventh effective amount of oxygen and a seventh effective amount of nitrous oxide to the patient for a seventh predetermined amount of time.

In addition, after administering the seventh effective amount of oxygen and the seventh effective amount of nitrous oxide to the patient for the seventh predetermined amount of time, methods can include administering an eighth effective amount of oxygen and an eighth effective amount of nitrous oxide to the patient for an eighth predetermined amount of time. Also, after administering the eighth effective amount of oxygen and the eighth effective amount of nitrous oxide to the patient for the eighth predetermined amount of time, methods can include administering a ninth effective amount of oxygen and a ninth effective amount of nitrous oxide to the patient for a ninth predetermined amount of time.

After administering the ninth effective amount of oxygen and the ninth effective amount of nitrous oxide to the patient for the ninth predetermined amount of time, methods can include administering a tenth effective amount of oxygen and a tenth effective amount of nitrous oxide to the patient for a tenth predetermined amount of time. After administering the tenth effective amount of oxygen and the tenth effective amount of nitrous oxide to the patient for the tenth predetermined amount of time, methods can include administering an eleventh effective amount of oxygen and an eleventh effective amount of nitrous oxide to the patient for an eleventh predetermined amount of time.

Even still, after administering the eleventh effective amount of oxygen and the eleventh effective amount of nitrous oxide to the patient for the eleventh predetermined amount of time, methods can include administering a twelfth effective amount of oxygen and a twelfth effective amount of nitrous oxide to the patient for a twelfth predetermined amount of time.

In some embodiments, the first predetermined amount of time is 20 minutes, the second predetermined amount of time is 2 minutes, the third predetermined amount of time is 2 minutes, the fourth predetermined amount of time is 2 minutes, the fifth predetermined amount of time is 2 minutes, the sixth predetermined amount of time is 2 minutes, the seventh predetermined amount of time is 2 minutes, the eighth predetermined amount of time is 2 minutes, the ninth predetermined amount of time is 2 minutes, the tenth predetermined amount of time is 2 minutes, the eleventh predetermined amount of time is 2 minutes, and the twelfth predetermined amount of time is 20 minutes. In some embodiments, the first effective amount of oxygen is 100%, the first effective amount of nitrous oxide is 0%, the second effective amount of oxygen is 90%, the second effective amount of nitrous oxide is 10%, the third effective amount of oxygen is 80%, the third effective amount of nitrous oxide is 20%, the fourth effective amount of oxygen is 70%, the a fourth effective amount of nitrous oxide is 30%, the fifth effective amount of oxygen is 60%, the fifth effective amount of nitrous oxide is 40%, the sixth effective amount of oxygen is 55%, the sixth effective amount of nitrous oxide is 45%, the seventh effective amount of oxygen is 50%, the seventh effective amount of nitrous oxide is 50%, the eighth effective amount of oxygen is 45%, the eighth effective amount of nitrous oxide is 55%, the ninth effective amount of oxygen is 40%, the ninth effective amount of nitrous oxide is 60%, the tenth effective amount of oxygen is 35%, the tenth effective amount of nitrous oxide is 65%, the eleventh effective amount of oxygen is 30%, the eleventh effective amount of nitrous oxide is 70%, the twelfth effective amount of oxygen is 100%, and the twelfth effective amount of nitrous oxide is 0%.

Methods can also include administering the first effective amount of oxygen starting at a predetermined rate and adjusting the predetermined rate according to a breathing rhythm of the patient. In some embodiments, the predetermined rate is 9.9 liters of oxygen per minute.

The effective amount of oxygen and the effective amount of nitrous oxide can be administered to the patient via a nitrous oxide sedation system. Some methods include coupling a portion of the nitrous oxide sedation system to a nose of the patient.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a view of a nitrous oxide sedation system coupled to a patient, according to some embodiments.

FIG. 2 illustrates a flow chart of a method for treating a patient suffering from alcohol withdrawal, according to some embodiments.

FIG. 3 illustrates a flow chart of another method for treating a patient suffering from alcohol withdrawal, according to some embodiments.

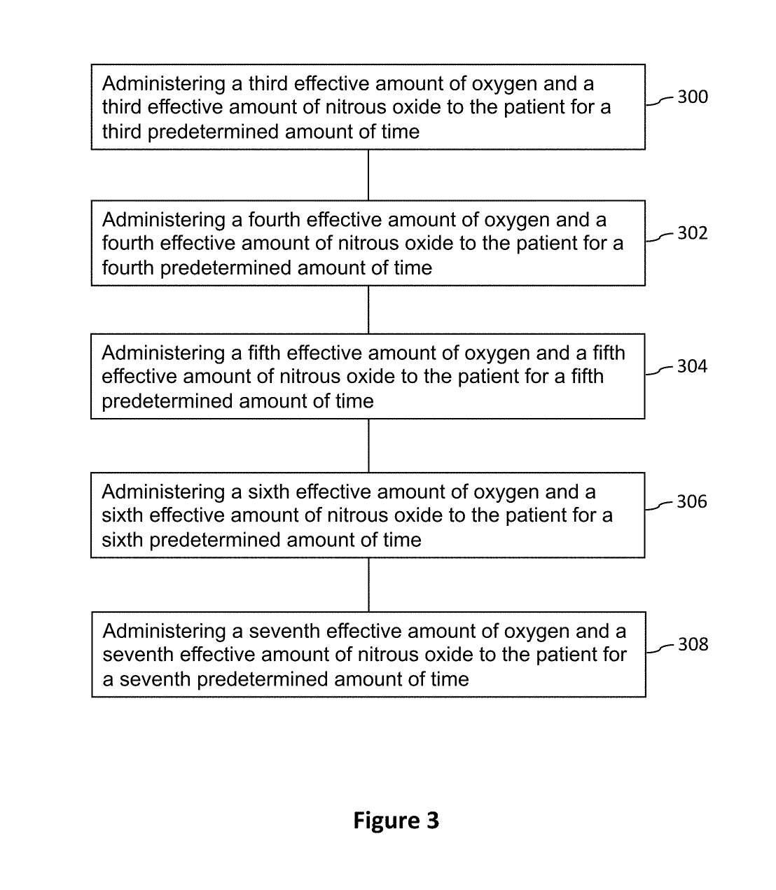

FIG. 4 illustrates a flow chart of another method for treating a patient suffering from alcohol withdrawal, according to some embodiments.

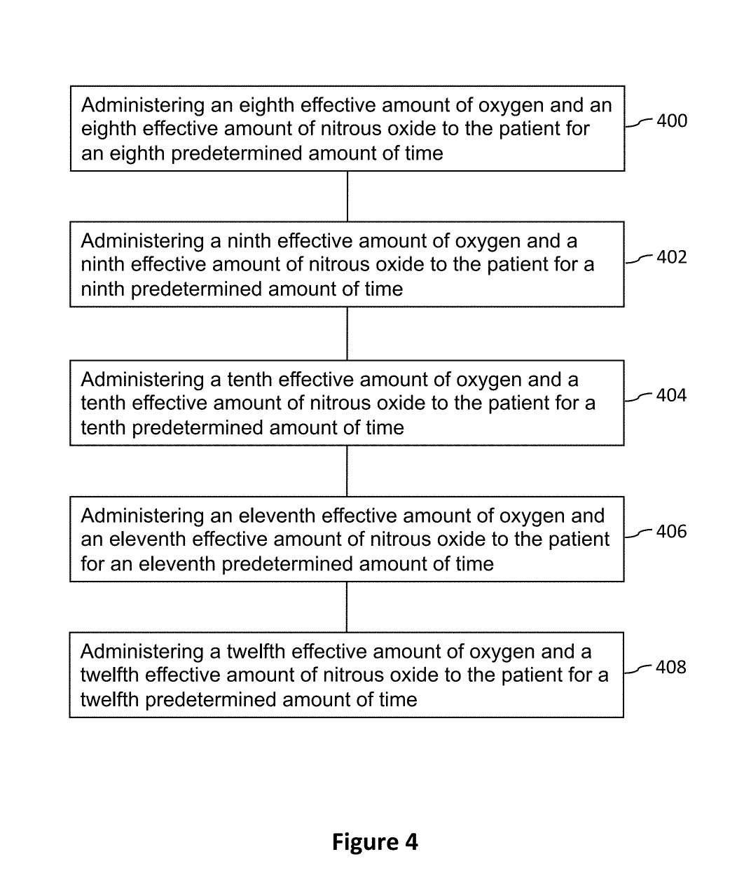

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Introduction

Alcohol consumption can effect how the brain functions, causing short-term and long-term changes. A person becomes addicted to alcohol when his or her brain adjusts to the way alcohol alters the brain so that drinking alcohol becomes necessary in order to function. Consequently, when the person does not consume alcohol their brain becomes excited and needs the alcohol slowing effects in order to function properly.

Alcohol also affects levels of endorphins, such as dopamine and serotonin. For example, alcohol increases the release of dopamine in your brain's “reward center”. By increasing the dopamine levels in your brain, alcohol tricks you into thinking that it is actually making you feel great. The effect is that you keep drinking to get more dopamine release, but at the same time you negatively alter other brain chemicals, such as serotonin that affects mood, behavior, thoughts, and speech. When a person consumes alcohol, serotonin levels are affected and bodily movements are slowed. And the more alcohol a person consumes, the more intense the effect. Accordingly, alcohol affects brain chemistry by altering levels of neurotransmitters, which control thought processes, behaviors, and emotions.

However, the adverse effects of alcohol on the brain can be reversed by introducing oxygen and nitrous oxide into the suffering patient. The nitrous oxide can cleanse the effected neurotransmitters, which can allow the parts of the neurons used to process alcohol to be restored to their normal function. The result of the treatment can have many benefits, such as no cravings for alcohol, no withdrawal signs, a clearer mind, better sense of sight and smell, a more positive outlook on life, and improved self-esteem.

### Treatment Methods

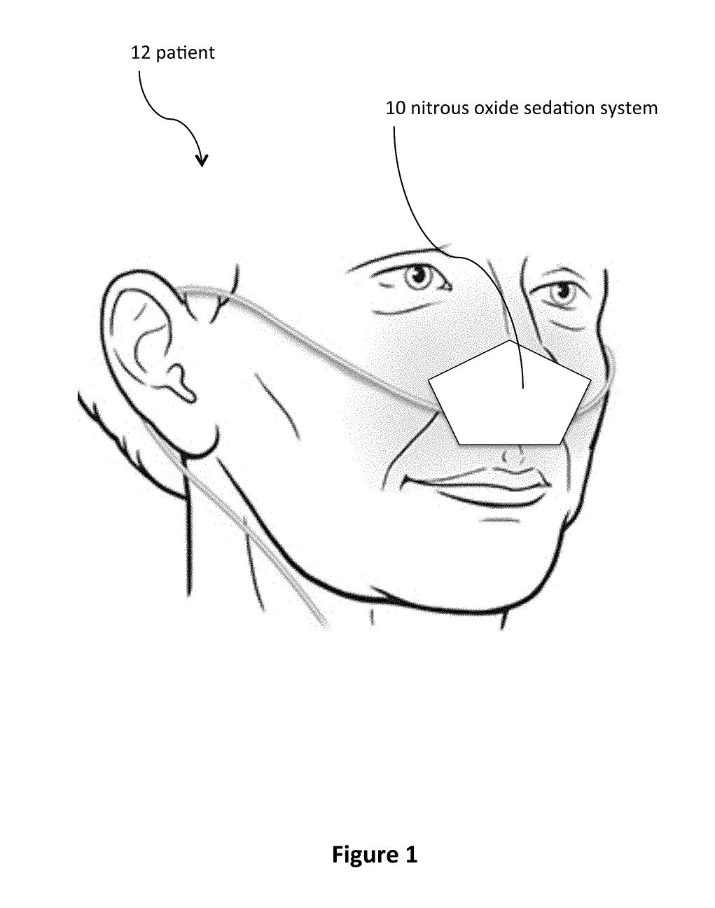

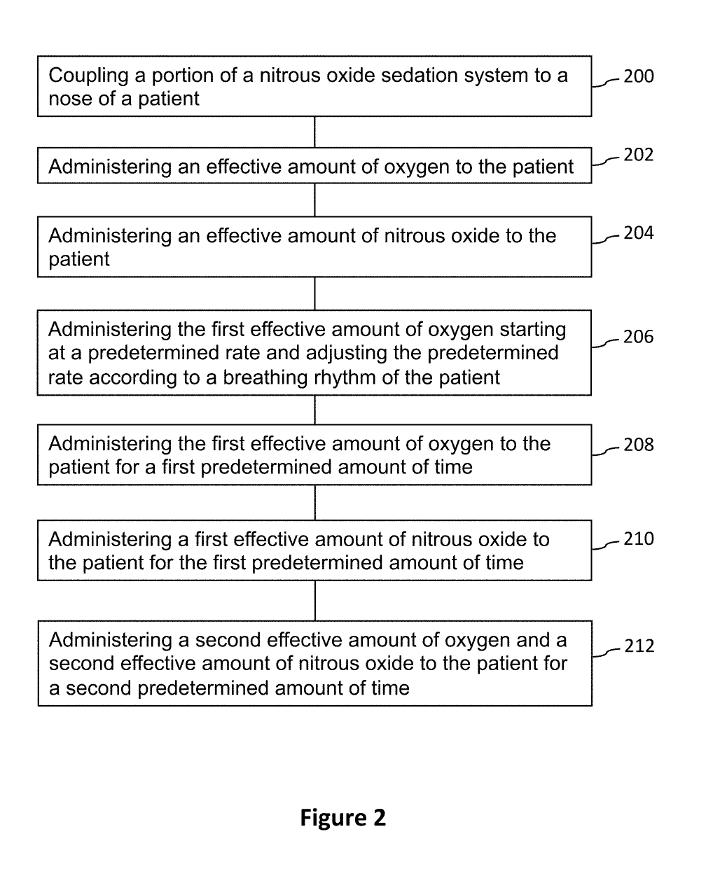

The present disclosure includes methods for treating a patient suffering from alcohol withdrawal. As shown in FIGS. 1 and 2, the method includes coupling a portion of a nitrous oxide sedation system 10 to a nose of a patient 12 (at step 200). The method also includes administering an effective amount of oxygen to the patient (at step 202) and administering an effective amount of nitrous oxide to the patient (at step 204).

With reference to FIG. 2, the method can include administering the first effective amount of oxygen starting at a predetermined rate and adjusting the predetermined rate according to a breathing rhythm of the patient (at step 206). In some embodiments, the predetermined rate is 9.9 liters of oxygen per minute. However, it should be appreciated that the predetermined rate can be any flow of oxygen less than or greater than 9.9 liters per minute.

The method can include administering a first effective amount of oxygen to the patient for a first predetermined amount of time (at step 208). In some embodiments, the first predetermined amount of time is 20 minutes and the first effective amount of oxygen is 100%. It should be appreciated that the first predetermined amount of time can be any amount of time less than or greater than 20 minutes. Additionally, the first effective amount of oxygen can be any percentage less than 100%.

Additionally, methods can include administering the effective amount of nitrous oxide to the patient comprises administering a first effective amount of nitrous oxide to the patient for the first predetermined amount of time (at step 210). In some embodiments, the first predetermined amount of time is 20 minutes and the first effective amount of nitrous oxide is 0%. It should be appreciated that the first predetermined amount of time can be any amount of time less than or greater than 20 minutes. Additionally, the first effective amount of nitrous oxide can be any percentage greater than 0%.

As previously disclosed, the method includes administering nitrous oxide to the patient to help alleviate the symptoms of alcohol withdrawal. Accordingly, after steps 208 and 210, the method can also include administering a second effective amount of oxygen and a second effective amount of nitrous oxide to the patient for a second predetermined amount of time (at step 212). In some embodiments, the second predetermined amount of time is 2 minutes, the second effective amount of oxygen is 90%, and the second effective amount of nitrous oxygen is 10%. It should be appreciated that the second predetermined amount of time can be any amount of time less than or greater than 2 minutes. Additionally, the second effective amount of oxygen can be any percentage less than or greater than 90%, while the second effective amount of nitrous oxide can be any percentage less than or greater than 10%.

The method can include administering a third effective amount of oxygen and a third effective amount of nitrous oxide to the patient for a third predetermined amount of time (at step 300). In some embodiments, the third predetermined amount of time is 2 minutes, the third effective amount of oxygen is 80%, and the third effective amount of nitrous oxygen is 20%. It should be appreciated that the third predetermined amount of time can be any amount of time less than or greater than 2 minutes. Additionally, the third effective amount of oxygen can be any percentage less than or greater than 80%, while the third effective amount of nitrous oxide can be any percentage less than or greater than 20%.

In some embodiments, the method includes administering a fourth effective amount of oxygen and a fourth effective amount of nitrous oxide to the patient for a fourth predetermined amount of time (at step 302). In some embodiments, the fourth predetermined amount of time is 2 minutes, the fourth effective amount of oxygen is 70%, and the fourth effective amount of nitrous oxygen is 30%. It should be appreciated that the fourth predetermined amount of time can be any amount of time less than or greater than 2 minutes. Additionally, the fourth effective amount of oxygen can be any percentage less than or greater than 70%, while the fourth effective amount of nitrous oxide can be any percentage less than or greater than 30%.

Methods can also include administering a fifth effective amount of oxygen and a fifth effective amount of nitrous oxide to the patient for a fifth predetermined amount of time (at step 304). In some embodiments, the fifth predetermined amount of time is 2 minutes, the fifth effective amount of oxygen is 60%, and the fifth effective amount of nitrous oxygen is 40%. It should be appreciated that the fifth predetermined amount of time can be any amount of time less than or greater than 2 minutes. Additionally, the fifth effective amount of oxygen can be any percentage less than or greater than 60%, while the fifth effective amount of nitrous oxide can be any percentage less than or greater than 40%.

The method can include administering a sixth effective amount of oxygen and a sixth effective amount of nitrous oxide to the patient for a sixth predetermined amount of time (at step 306). In some embodiments, the sixth predetermined amount of time is 2 minutes, the sixth effective amount of oxygen is 55%, and the sixth effective amount of nitrous oxygen is 45%. It should be appreciated that the sixth predetermined amount of time can be any amount of time less than or greater than 2 minutes. Additionally, the sixth effective amount of oxygen can be any percentage less than or greater than 55%, while the sixth effective amount of nitrous oxide can be any percentage less than or greater than 45%.

In some embodiments, the method includes administering a seventh effective amount of oxygen and a seventh effective amount of nitrous oxide to the patient for a seventh predetermined amount of time (at step 308). In some embodiments, the seventh predetermined amount of time is 2 minutes, the seventh effective amount of oxygen is 50%, and the seventh effective amount of nitrous oxygen is 50%. It should be appreciated that the seventh predetermined amount of time can be any amount of time less than or greater than 2 minutes. Additionally, the seventh effective amount of oxygen can be any percentage less than or greater than 50%, while the seventh effective amount of nitrous oxide can be any percentage less than or greater than 50%.

Methods can also include administering an eighth effective amount of oxygen and an eighth effective amount of nitrous oxide to the patient for an eighth predetermined amount of time (at step 400). In some embodiments, the eighth predetermined amount of time is 2 minutes, the eighth effective amount of oxygen is 45%, and the eighth effective amount of nitrous oxygen is 55%. It should be appreciated that the eighth predetermined amount of time can be any amount of time less than or greater than 2 minutes. Additionally, the eighth effective amount of oxygen can be any percentage less than or greater than 45%, while the eighth effective amount of nitrous oxide can be any percentage less than or greater than 55%.

The method can include administering a ninth effective amount of oxygen and a ninth effective amount of nitrous oxide to the patient for a ninth predetermined amount of time (at step 402). In some embodiments, the ninth predetermined amount of time is 2 minutes, the ninth effective amount of oxygen is 40%, and the ninth effective amount of nitrous oxygen is 60%. It should be appreciated that the ninth predetermined amount of time can be any amount of time less than or greater than 2 minutes. Additionally, the ninth effective amount of oxygen can be any percentage less than or greater than 40%, while the ninth effective amount of nitrous oxide can be any percentage less than or greater than 60%.

In some embodiments, the method includes administering a tenth effective amount of oxygen and a tenth effective amount of nitrous oxide to the patient for a tenth predetermined amount of time (at step 404). In some embodiments, the tenth predetermined amount of time is 2 minutes, the tenth effective amount of oxygen is 35%, and the tenth effective amount of nitrous oxygen is 65%. It should be appreciated that the tenth predetermined amount of time can be any amount of time less than or greater than 2 minutes. Additionally, the tenth effective amount of oxygen can be any percentage less than or greater than 35%, while the tenth effective amount of nitrous oxide can be any percentage less than or greater than 65%.

Methods can also include administering an eleventh effective amount of oxygen and an eleventh effective amount of nitrous oxide to the patient for an eleventh predetermined amount of time (at step 406). In some embodiments, the eleventh predetermined amount of time is 2 minutes, the eleventh effective amount of oxygen is 30%, and the eleventh effective amount of nitrous oxygen is 70%. It should be appreciated that the eleventh predetermined amount of time can be any amount of time less than or greater than 2 minutes. Additionally, the eleventh effective amount of oxygen can be any percentage less than or greater than 30%, while the eleventh effective amount of nitrous oxide can be any percentage less than or greater than 70%.

The method can include administering a twelfth effective amount of oxygen and a twelfth effective amount of nitrous oxide to the patient for a twelfth predetermined amount of time (at step 408). In some embodiments, the twelfth predetermined amount of time is 20 minutes, the twelfth effective amount of oxygen is 100%, and the twelfth effective amount of nitrous oxygen is 0%. It should be appreciated that the twelfth predetermined amount of time can be any amount of time less than or greater than 20 minutes. Additionally, the twelfth effective amount of oxygen can be any percentage less than 100%, while the twelfth effective amount of nitrous oxide can be any percentage greater than 0%.

### Interpretation

None of the steps or limitations described herein is essential or indispensable. Any of the steps or limitations can be adjusted or modified. Other or additional steps and/or limitations can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

Some of the devices, systems, embodiments, and processes use computers. Each of the routines, processes, methods, and algorithms described in the preceding sections may be embodied in, and fully or partially automated by, code modules executed by one or more computer, processor, or machine configured to execute computer instructions. The code modules may be stored on any type of non-transitory computer-readable storage medium or tangible computer storage device, such as a hard drive solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

The following is claimed:

1. A method for treating a patient suffering from alcohol withdrawal, comprising:
administering an effective amount of oxygen to the patient; and
administering an effective amount of nitrous oxide to the patient.

2. The method of claim 1, wherein administering the effective amount of oxygen to the patient comprises administering a first effective amount of oxygen to the patient for a first predetermined amount of time, and wherein administering the effective amount of nitrous oxide to the patient comprises administering a first effective amount of nitrous oxide to the patient for the first predetermined amount of time.

3. The method of claim 2, further comprising, then after administering the first effective amount of oxygen and the first effective amount of nitrous oxide to the patient for the first predetermined amount of time, administering a second effective amount of oxygen and a second effective amount of nitrous oxide to the patient for a second predetermined amount of time.

4. The method of claim 3, further comprising, then after administering the second effective amount of oxygen and the second effective amount of nitrous oxide to the patient for the second predetermined amount of time, administering a third effective amount of oxygen and a third effective amount of nitrous oxide to the patient for a third predetermined amount of time.

5. The method of claim 4, further comprising, then after administering the third effective amount of oxygen and the third effective amount of nitrous oxide to the patient for the third predetermined amount of time, administering a fourth effective amount of oxygen and a fourth effective amount of nitrous oxide to the patient for a fourth predetermined amount of time.

6. The method of claim 5, further comprising, then after administering the fourth effective amount of oxygen and the fourth effective amount of nitrous oxide to the patient for the fourth predetermined amount of time, administering a fifth effective amount of oxygen and a fifth effective amount of nitrous oxide to the patient for a fifth predetermined amount of time.

7. The method of claim 6, further comprising, then after administering the fifth effective amount of oxygen and the fifth effective amount of nitrous oxide to the patient for the fifth predetermined amount of time, administering a sixth effective amount of oxygen and a sixth effective amount of nitrous oxide to the patient for a sixth predetermined amount of time.

8. The method of claim 7, further comprising, then after administering the sixth effective amount of oxygen and the sixth effective amount of nitrous oxide to the patient for the sixth predetermined amount of time, administering a seventh effective amount of oxygen and a seventh effective amount of nitrous oxide to the patient for a seventh predetermined amount of time.

9. The method of claim 8, further comprising, then after administering the seventh effective amount of oxygen and the seventh effective amount of nitrous oxide to the patient for the seventh predetermined amount of time, administering an eighth effective amount of oxygen and an eighth effective amount of nitrous oxide to the patient for an eighth predetermined amount of time.

10. The method of claim 9, further comprising, then after administering the eighth effective amount of oxygen and the eighth effective amount of nitrous oxide to the patient for the eighth predetermined amount of time, administering a ninth effective amount of oxygen and a ninth effective amount of nitrous oxide to the patient for a ninth predetermined amount of time.

11. The method of claim 10, further comprising, then after administering the ninth effective amount of oxygen and the ninth effective amount of nitrous oxide to the patient for the ninth predetermined amount of time, administering a tenth effective amount of oxygen and a tenth effective amount of nitrous oxide to the patient for a tenth predetermined amount of time.

12. The method of claim 11, further comprising, then after administering the tenth effective amount of oxygen and the tenth effective amount of nitrous oxide to the patient for the tenth predetermined amount of time, administering an eleventh effective amount of oxygen and an eleventh effective amount of nitrous oxide to the patient for an eleventh predetermined amount of time.

13. The method of claim 12, further comprising, then after administering the eleventh effective amount of oxygen and the eleventh effective amount of nitrous oxide to the patient for the eleventh predetermined amount of time, administering a twelfth effective amount of oxygen and a twelfth effective amount of nitrous oxide to the patient for a twelfth predetermined amount of time.

14. The method of claim 13, wherein the first predetermined amount of time is 20 minutes, the second predetermined amount of time is 2 minutes, the third predetermined amount of time is 2 minutes, the fourth predetermined amount of time is 2 minutes, the fifth predetermined amount of time is 2 minutes, the sixth predetermined amount of time is 2 minutes, the seventh predetermined amount of time is 2 minutes, the eighth predetermined amount of time is 2 minutes, the ninth predetermined amount of time is 2 minutes, the tenth predetermined amount of time is 2 minutes, the eleventh predetermined amount of time is 2 minutes, and the twelfth predetermined amount of time is 20 minutes.

15. The method of claim 14, wherein the first effective amount of oxygen is 100%, the first effective amount of nitrous oxide is 0%, the second effective amount of oxygen is 90%, the second effective amount of nitrous oxide is 10%, the third effective amount of oxygen is 80%, the third effective amount of nitrous oxide is 20%, the fourth effective amount of oxygen is 70%, the a fourth effective amount of nitrous oxide is 30%, the fifth effective amount of oxygen is 60%, the fifth effective amount of nitrous oxide is 40%, the sixth effective amount of oxygen is 55%, the sixth effective amount of nitrous oxide is 45%, the seventh effective amount of oxygen is 50%, the seventh effective amount of nitrous oxide is 50%, the eighth effective amount of oxygen is 45%, the eighth effective amount of nitrous oxide is 55%, the ninth effective amount of oxygen is 40%, the ninth effective amount of nitrous oxide is 60%, the tenth effective amount of oxygen is 35%, the tenth effective amount of nitrous oxide is 65%, the eleventh effective amount of oxygen is 30%, the eleventh effective amount of nitrous oxide is 70%, the twelfth effective amount of oxygen is 100%, and the twelfth effective amount of nitrous oxide is 0%.

16. The method of claim 2, further comprising administering the first effective amount of oxygen starting at a predetermined rate and adjusting the predetermined rate according to a breathing rhythm of the patient.

17. The method of claim 16, wherein the predetermined rate is 9.9 liters of oxygen per minute.

18. The method of claim 1, wherein the effective amount of oxygen and the effective amount of nitrous oxide are administered to the patient via a nitrous oxide sedation system.

19. The method of claim 18, further comprising coupling a portion of the nitrous oxide sedation system to a nose of the patient.
